(19) United States
(12) Patent Application Publication
(10) Pub. No.: US 2018/0155270 A1
(43) Pub. Date: Jun. 7, 2018

(54) PROCESS FOR PREPARATION OF CINACALCET INTERMEDIATE AND CINACALCET HYDROCHLORIDE

(71) Applicant: Lupin Limited, Munbai (IN)

(72) Inventors: Vishal Gautam Gaikwad, Pune (IN); Sadanand Nilkanth Patil, Pune (IN); Rajinder Singh Siyan, Pune (IN); Nandu Baban Bhise, Pune (IN); Girij Pal Singh, Pune (IN)

(21) Appl. No.: 15/577,365

(22) PCT Filed: May 27, 2016
(86) PCT No.: PCT/IB2016/053127

§ 371 Date: Nov. 28, 2017

(30) Foreign Application Priority Data
May 29, 2015 (IN) 2093/MUM/2015

(51) Int. Cl. C07C 209/08; C07C 211/27; C07C 211/30; C07C 29/147; C07C 31/38; C07C 17/16; C07C 22/08; C07C 5/03

(52) U.S. Cl. CPC C07C 209/08; C07C 211/27; C07C 211/30; C07C 29/147; C07C 31/38; C07C 2525/02; C07C 22/08; C07C 5/03; C07C 2523/42; C07C 2523/44; C07C 17/16

(57) ABSTRACT

The present invention provides one pot process for preparation of highly pure unsaturated cinacalcet hydrochloride (II) comprising: i) converting 3-(trifluromethyl) cinnamic acid (III) into 3-(3-(trifluoromethyl)phenyl)prop-2-en-1-ol (IV), ii) converting 3-(3-(trifluoromethyl)phenyl)prop-2-en-1-ol (IV) to compound (V), wherein R is Cl, Br, I, tosylate and mesylate, Formula (V) iii) reacting compound (V) with (R)-1-(1-Naphthyl) ethylamine (VI) in presence of base followed by treatment with hydrochloric acid. The present invention further provides conversion of unsaturated cinacalcet hydrochloride (II) to cinacalcet hydrochloride (I).

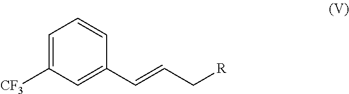

### FIELD OF INVENTION

The present invention provides one pot process for preparation of highly pure unsaturated cinacalcet hydrochloride (II), which is used for preparation of cinacalcet hydrochloride (I) thereof.

### BACKGROUND OF THE INVENTION

Cinacalcet hydrochloride (I) is a calcium-sensing receptor agonist indicated for secondary hyperparathyroidism in patients with chronic kidney disease and for hypercalcemia in patients with parathyroid carcinoma. It is chemically known as N-[1-(R)-(−)-(1-naphthyl)ethyl]-3-[3(trifluoromethyl)phenyl]-1-aminopropane hydrochloride and is represented by structural formula given below.

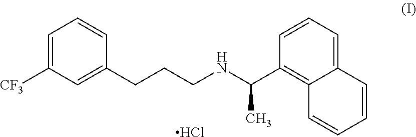 [SMILES: C[C@@H](NCCCc1cccc(C(F)(F)F)c1)c1cccc2ccccc12 ; Cl]

Cinacalcet hydrochloride (I) is specifically disclosed in U.S. Pat. No. 6,211,244 and this patent provides process for preparation of structurally analogues compounds only. The article Drugs of the future 2002, 27 (9), 831-836, in scheme I provides process for preparation of cinacalcet, which involves reaction of 3-[3-(trifluoromethyl)phenyl]propionaldehyde prepared by Swern oxidation of the corresponding alcohol with (R)-1-(1-naphthyl)ethylamine in the presence of titanium isopropoxide to give imine which upon reduction gives cinacalcet base. This process involves the use of highly inflammable and toxic titanium isopropoxide.

The U.S. Pat. No. 8,759,586 provides process for preparation of cinacalcet which comprises converting the hydroxyl moiety of 3-(3-(trifluoromethyl)phenyl)prop-2-en-1-ol into a good leaving group and then reacting it with (R)-1-naphthylethylamine in presence of base, followed by treatment with hydrochloric acid to give unsaturated cinacalcet hydrochloride (II) and reducing it to obtain cinacalcet hydrochloride. The process involves isolation of all the intermediates which makes the process tedious and time consuming

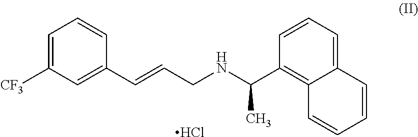 [SMILES: C[C@@H](NC/C=C/c1cccc(C(F)(F)F)c1)c1cccc2ccccc12 ; Cl]

The present invention provides one pot process for preparation of highly pure unsaturated cinacalcet hydrochloride (II), process is simple, commercially viable, avoids multiple steps of isolation and purification of the precursors. Unsaturated cinacalcet hydrochloride (II) is an important intermediate for preparation of cinacalcet hydrochloride (I).

### SUMMARY OF THE INVENTION

The present invention provides one pot process for preparation of highly pure unsaturated cinacalcet hydrochloride (II) comprising:
i) converting 3-(trifluromethyl) cinnamic acid (III) into 3-(3-(trifluoromethyl)phenyl)prop-2-en-1-ol (IV),
ii) converting 3-(3-(trifluoromethyl)phenyl)prop-2-en-1-ol (IV) to compound (V), wherein R is Cl, Br, I, tosylate and mesylate,

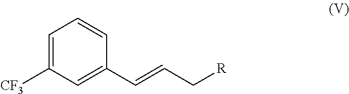

iii) reacting compound (V) with (R)-1-(1-Naphthyl) ethylamine (VI) in presence of base followed by treatment with hydrochloric acid.

The present invention further provides conversion of unsaturated cinacalcet hydrochloride (II) to cinacalcet hydrochloride (I).

### DETAILED DESCRIPTION OF THE INVENTION

In one embodiment, the present invention provides one pot process for preparation of highly pure unsaturated cinacalcet hydrochloride (II) comprising:
i) converting 3-(trifluromethyl)cinnamic acid (III) into 3-(3-(trifluoromethyl)phenyl)prop-2-en-1-ol (IV),

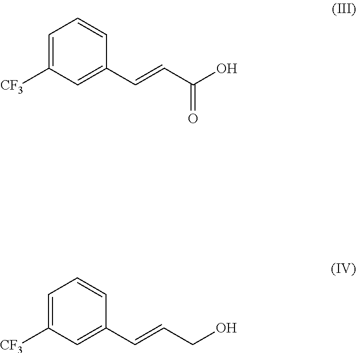 [SMILES: O=C(O)/C=C/c1cccc(C(F)(F)F)c1 ; OC/C=C/c1cccc(C(F)(F)F)c1]

ii) converting 3-(3-(trifluoromethyl)phenyl)prop-2-en-1-ol (IV) to compound (V), wherein R is Cl, Br, I, tosylate and mesylate

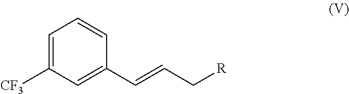

iii) reacting compound (V) with (R)-1-(1-Naphthyl) ethylamine (VI) in presence of base followed by treatment with hydrochloric acid

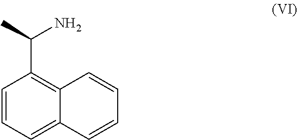

In another embodiment, the present invention provides conversion of unsaturated cinacalcet hydrochloride (II) to cinacalcet hydrochloride (I).

The compound 3-(trifluromethyl) cinnamic acid (III) is commercially available or can be prepared by methods known in literature.

The compound 3-(trifluromethyl) cinnamic acid (III) can be converted to 3-(3-(trifluoromethyl)phenyl)prop-2-en-1-ol (IV) by reaction of 3-(trifluromethyl) cinnamic acid (III) with ethylchloroformate followed by reaction with sodium borohydride.

The reaction of 3-(trifluromethyl) cinnamic acid (III) and ethylchloroformate can be carried out in presence of organic base like alkylamine selected from diethylamine, triethylamine diisopropylamine preferably triethylamine and inert solvent like ethers selected form tetrahydrofuran, diethyl ether, diisopropyl ether, methyl t-butyl ether, di-tert-butyl ether, diglyme, preferably tetrahydrofuran.

The compound 3-(3-(trifluoromethyl)phenyl)prop-2-en-1-ol (IV) is converted to compound (V), wherein R is Cl, Br, I, tosylate or mesylate, by reacting compound (IV) with a suitable reagent and a solvent. Suitable reagent is selected form thionyl halide, aliphatic sulfonyl halide or aromatic sulfonyl halide like thionyl chloride, thionyl bromide, methanesulfonyl chloride, benzenesulfonyl chloride, 4-nitobenzensulfonylchloride, or p-toluenesulfonyl chloride. Compound (V) wherein R is Cl, can be obtained by reaction of 3-(3-(trifluoromethyl) phenyl) prop-2-en-1-ol (IV) with hydrochloric acid in a solvent. The reaction can be carried out in solvents selected from chlorinated hydrocarbon like dichloromethane, dichloroethane; ethers likes tetrahydrofuran, diethyl ether, diisopropyl ether, methyl t-butyl ether, di-tert-butyl ether, diglyme, preferably tetrahydrofuran; aromatic hydrocarbon like toluene, xylene; acetonitrile or a mixture thereof; preferably dichloromethane.

The reaction of compound (V) with (R)-1-(1-Naphthyl) ethylamine (VI) is carried out in presence of inorganic base selected from hydroxide, alkoxides carbonates, bicarbonates of alkali or alkaline earth metal like sodium hydroxide, potassium hydroxide, sodium methoxide, potassium methoxide, sodium carbonate, potassium carbonate, sodium bicarbonate; preferably potassium carbonate. The reaction can be carried out in solvents selected from chlorinated hydrocarbon like dichloromethane, dichloroethane; ethers likes diethylether, diisopropyl ether, tetrahydrofuran; aromatic hydrocarbon like toluene, xylene; acetonitrile or a mixture thereof; preferably dichloromethane.

Unsaturated cinacalcet hydrochloride (II) is an important intermediate for preparation of cinacalcet hydrochloride. The present invention provides one pot process for preparation of compound (II) which is highly pure. The term “highly pure” refers to compound (II) with HPLC purity of greater than 99%, more preferably greater than 99.9%.

The present invention further provides conversion of highly pure unsaturated cinacalcet hydrochloride (II) to cinacalcet hydrochloride (I).

Unsaturated cinacalcet hydrochloride (II) can be reduced by catalytic hydrogenation to give cinacalcet hydrochloride (I). The catalytic hydrogenation can be carried out under H₂ pressure in presence of catalyst such as Pd/C, PtO₂ or Raney nickel. The reaction can be carried out in an alcohol solvent selected form methanol, ethanol, isopropanol, butanol or mixtures thereof.

Cinacalcet hydrochloride (I) obtained by the present process has HPLC purity of greater than 99%, preferably greater than 99.9%.

Thus the present invention provides simple and commercial process for preparation of highly pure cinacalcet intermediate (II) and cinacalcet hydrochloride (I) thereof.

The present invention is further illustrated by the following representative examples and does not limit the scope of the invention.

### Example 1: Preparation of Unsaturated Cinacalcet Hydrochloride (II)

A mixture of 3-(trifluromethyl) cinnamic acid (100 g), triethylamine (47 g) and tetrahydrofuran (600 ml) was cooled to −20 to −15° C. Ethylchloroformate (55 g) was added to the mixture and stirred for one hour at −20 to −15° C. The reaction mixture was filtered and the filtrate was collected. To the filtrate was added pre-cooled sodium borohydride solution (45 g sodium borohydride and 0.45 g of sodium hydroxide in 510 ml water) at 0-5° C. The reaction mixture was stirred for 2 hours at 20-30° C. To the reaction mixture was added 5% aqueous hydrochloride solution (500 ml), dichloromethane (1000 ml) and water (500 ml). The organic layer was separated and was concentrated under vacuum. To the residue was added concentrated hydrochloride acid (500 ml) and the mixture was stirred at 20-25° C. for 1 hour. Dichloromethane (1000 ml) was added to the mixture. The organic layer separated and concentrated under vacuum. To the residue was added water (1000 ml), potassium carbonate (126 g) and (R)-1-(1-Naphthyl) ethylamine (71 g) and reaction mass stirred at 80-85° C. for about 6 hours. The reaction mixture cooled to 20-30° C. and dichloromethane (1000 ml) was added, followed by addition of concentrated hydrochloric acid (170 ml). The organic layer was separated, washed with water (1000 ml) and then concentrated under vacuum and n-heptane (1100 ml) was added to the residue. Solid was obtained which was filtered and washed with n-heptane (200 ml). The solid was taken up in dichloromethane (1000 ml) and washed with water (500 ml). The organic layer was separated and concentrated under vacuum to residual 2-3 volumes and n-heptane (300 ml) was added and the mixture was again concentrated under vacuum to residual 2-3 volumes and n-heptane (800 ml) was added. The mixture was stirred for 2 hours at 20-25° C. and the solid was filtered, washed with n-heptane (400 ml) and dried under vacuum. Yield: 61%; HPLC purity: 99.9%.

### Example 2: Preparation of Cinacalcet Hydrochloride (II)

To a mixture of unsaturated cinacalcet hydrochloride (II, 100 g) and methanol (500 ml) was added 10% potassium carbonate (1.5 ml) to adjust pH of the reaction mixture to 4.1. To the mixture was added 10% Pd/C and hydrogen gas was purged. Hydrogen pressure was maintained at 2-3 kg/cm² and the mixture was stirred at 20-25° C. for about 3 hours. The reaction mass filtered, washed with methanol and the filtrate was concentrated under vacuum to residual 2-3 volumes. To the residue was added dichloromethane (1000 ml) and water (500 ml). The organic layer was separated and concentrated to residual 2-3 volumes and acetonitrile (200 ml) was added and concentrated further to residual 2-3 volumes and acetonitrile (400 ml) was again added. The mixture was stirred at 80-85° C. for 10 minutes and then cooled to 10-15° C. The solid was filtered and dried under vacuum. The solid was crystallized from mixture of acetonitrile and water. Yield: 71%; HPLC purity: 100%.

1. A one pot process for preparation of highly pure unsaturated cinacalcet hydrochloride (II) comprising:
i) converting 3-(trifluromethyl)cinnamic acid (III) into 3-(3-(trifluoromethyl)phenyl)prop-2-en-1-ol (IV), 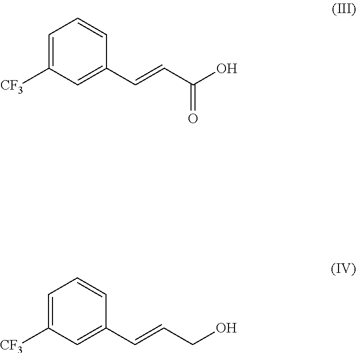 [SMILES: O=C(O)/C=C/c1cccc(C(F)(F)F)c1 ; OC/C=C/c1cccc(C(F)(F)F)c1]
ii) converting 3-(3-(trifluoromethyl)phenyl)prop-2-en-1-ol (IV) to compound (V), wherein R is Cl, Br, I, tosylate and mesylate 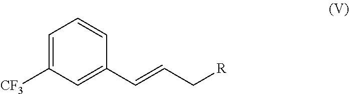
iii) reacting compound (V) with (R)-1-(1-Naphthyl) ethylamine (VI) in presence of base followed by treatment with hydrochloric acid

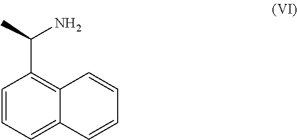

2. The process according to claim 1 wherein, conversion of 3-(trifluromethyl)cinnamic acid (III) to 3-(3-(trifluoromethyl)phenyl)prop-2-en-1-ol (IV), is carried out by using ethylchloroformate in presence of organic base followed by reaction with sodium borohydride.

3. The process according to claim 2 wherein, organic base is selected from alkylamine, selected from diethylamine, triethylamine, diisopropylamine.

4. The process according to claim 1 wherein, conversion of 3-(3-(trifluoromethyl)phenyl)prop-2-en-1-ol (IV) to compound (V), wherein R is CI, Br, I, tosylate and mesylate, is carried out by reacting compound (IV) with a suitable reagent and a solvent.

5. The process according to claim 4 wherein, suitable reagent is selected from thionyl halide, aliphatic sulfonyl halide or aromatic sulfonyl halide.

6. The process according to claim 5 wherein, suitable reagent is selected from thionyl chloride, thionyl bromide, methanesulfonyl chloride, benzenesulfonyl chloride, 4-nitobenzensulfonylchloride, p-toluenesulfonyl chloride.

7. The process according to claim 1 wherein, conversion of 3-(3-(trifluoromethyl)phenyl)prop-2-en-1-ol (IV) to compound (V), wherein R is CI, can be carried out in presence of hydrochloride acid in a solvent.

8. The process according to claims 4 and 7 wherein, solvent is selected from chlorinated hydrocarbon, ether, aromatic hydrocarbon and nitriles or a mixture thereof.

9. The process according to claim 8 wherein, solvent is selected from dichloromethane, dichloroethane, diethylether, diisopropyl ether, tetrahydrofuran, toluene, xylene, acetonitrile or a mixture thereof.

10. The process according to claim 1 wherein, compound (V) is reacted with (R)-1-(1-Naphthyl) ethylamine (VI) in presence of base.

11. The process according to claim 10 wherein, base is selected from sodium hydroxide, potassium hydroxide, sodium methoxide, potassium methoxide, sodium carbonate, potassium carbonate, sodium bicarbonate.

12. The process according to claim 1 wherein, unsaturated cinacalcet hydrochloride (II) has a HPLC purity of greater than 99%.

13. The process according to claim 1 wherein, unsaturated cinacalcet hydrochloride (II) has a HPLC purity of greater than 99.9%.

14. The process according to claim 1 further comprising, conversion of unsaturated cinacalcet hydrochloride (II) to cinacalcet hydrochloride (I).

15. The process according to claim 14 wherein, conversion of unsaturated cinacalcet hydrochloride (II) to cinacalcet hydrochloride (I) is carried out by catalytic hydrogenation.

16. The process according to claim 15 wherein, catalytic hydrogenation can be carried out under H₂ pressure in presence of catalyst selected from Pd/C, PtO₂, Raney nickel.

17. The process according to claim 15 wherein, catalytic hydrogenation can be carried out in an alcohol solvent selected form methanol, ethanol, isopropanol, butanol or mixtures thereof.

18. The process according to claim 7, wherein solvent is selected from chlorinated hydrocarbon, ether, aromatic hydrocarbon and nitriles or a mixture thereof.
